(19) United States
(12) Patent Application Publication
(10) Pub. No.: US 2004/0131587 A1
(43) Pub. Date: Jul. 8, 2004

(54) Method for treatment of tumors using combination therapy

(76) Inventors: Elaine K. Thomas, Seattle, WA (US); Stewart D. Lyman, Seattle, WA (US); David H. Lynch, Seattle, WA (US); Thibaut N. De Smedt, Seattle, WA (US); Charles R. Maliszewski, Seattle, WA (US)

Correspondence Address: IMMUNEX CORPORATION, LAW DEPARTMENT, 1201 AMGEN COURT WEST, SEATTLE, WA, 98119, US

(21) Appl. No.: 10/381,160

(22) PCT Filed:
(86) PCT No.: PCT/US01/46254

(51) Int. Cl. A61K 38/20; A61K 38/18

(52) U.S. Cl. 424/85.2; 514/12

(57) ABSTRACT

An improved method for treatment of a tumor bearing subject comprising administering to said subject a combination of from two to five agents is disclosed. The agents may be agents that mobilize dendritic cells, agents that cause apoptosis and/or necrosis of tumor cells, chemoattractants, agents that stimulate maturation of dendritic cells, and agents that enhance an anti-tumor response of a T cell.

### BACKGROUND OF THE INVENTION

1. Field of the Invention

The present invention relates to oncology therapeutic methods and more particularly relates to combination therapies that involve treating tumor bearing subjects with a combination of agents that collectively increase dendritic cells, stimulate the maturation of the dendritic cells and cause dendritic cells to present antigen to T cells, and stimulate the T cells.

2. Description of the Relevant Art

The term cancer covers a broad variety of disease states in which the normal growth of cells has been disrupted. Although there has been much progress in the treatment of cancer, some forms remain less amenable to treatment than others. One of the challenges in treatment arises because there are numerous types of cancers, which originate from various types of normal cells. It is generally thought that progression to disease occurs because the abnormal cells evade the immune system and proliferate uncontrollably. Thus, evasion of the immune system appears to be common for most, if not all, cancers.

The understanding that the immune system plays a critical role in development of cancer has sparked a great deal of interest in various means of stimulating the immune system to recognize cancerous cells and eliminate them. Various biological response modifiers have been investigated for anti-cancer therapeutic uses, including Interleukins 2, 4, and 6, and other cytokines. Although each factor may evince efficacy in some patients, not one has been shown to be broadly effective. Moreover, several such factors have been found to have dose-limiting toxic effects. Thus, investigators have been seeking combinations of various factors that will allow the immune response to develop effective anti-tumor activity with minimal deleterious effects. However, prior to the present invention, the optimal types of combinations were not known

### SUMMARY OF THE INVENTION

The present invention provides methods for treating a tumor-bearing subject by:

(a) administering a DC mobilization factor; (b) administering a tumor killing agent; and (c) administering a DC maturation agent. In one embodiment of the invention, the tumor killing agent is the same agent that stimulates maturation of dendritic cells (DC maturation agent). The methods described herein optionally further include the steps of administering one or more T lymphocyte enhancing agents, and/or administering a chemoattractant to attract mobilized dendritic cells and/or T cells to a specific site, such as a tumor site. Optionally, the methods may further include administering tumor antigen(s) to the subject.

In one embodiment, the methods present invention are in vivo combination immunotherapy methods in which the just described agents (DC mobilization factor, DC maturation agent, tumor killing agent, T lymphocyte enhancing agent, and chemoattractant) are administered to a tumor-bearing subject by any suitable method, including topically, subcutaneous, intravenous, intratumoral, intranodal or intramuscular administration, administration in the form of a controlled or sustained release formulation, oral administration, or use of any other route known to one of routine skill in the art. Moreover, the various agents may be administered locally, in or near the site of the tumor, for example by application of a localized sustained release formulation during or immediately after surgery, laser treatment, radiation therapy, viral infection of the tumor or other tumor-ablative therapy, or by use of other methods known in the art to deliver an agent or agents to a tumor site.

In another embodiment, the methods of the present invention are combination immunotherapy methods in which one or more of the above described administering steps is performed ex vivo. For example, the present invention provides combination therapies that include (a) administering a therapeutically effective amount of a DC mobilization factor to a tumor bearing subject; (b) obtaining dendritic cells from the tumor bearing subject administered a DC mobilization factor; (c) culturing the dendritic cells obtained from the tumor bearing subject in an ex vivo culture; and (d) administering the cultured dendritic cells to the tumor bearing subject. Preferably the dendritic cells are administered at a time when the anti-tumor therapy will not adversely affect the dendritic cells that are being administered.

Optionally, the ex vivo combination immunotherapy methods of the present invention further include the step of contacting the cultured dendritic cells with a tumor antigen in such a way that the cells are able to present the tumor antigen to other immune cells. Additionally the ex vivo methods may include the step of treating cultured dendritic cells with an agent that stimulates activation and/or maturation of dendritic cells in order to facilitate antigen presentation. The step of treating the cultured dendritic cells with an agent that stimulates activation and/or maturation of dendritic cells may be performed before or after contacting the cultured dendritic cells with the antigen, depending upon whether the antigen requires processing or not. Typically, if the antigen requires processing by the dendritic cell, treating the cultured dendritic cells is performed after the dendritic cells have processed the antigen. If the antigen does not require processing by the cultured dendritic cells, treating the cultured dendritic cells with an agent that stimulates activation and/or maturation of dendritic cells step is performed prior to contacting the cultured dendritic cells with antigen.

In yet another embodiment, the present invention further includes causing the dendritic cells to secrete certain cytokines. In ex vivo methods, this may be accomplished by contacting the dendritic cells with one or more agents that induce the cytokine expression, or by transfecting dendritic cells with a gene encoding the cytokines.

Concurrent with administering cultured dendritic cells to a tumor bearing individual the present invention further includes administering cultured dendritic cells or mature, antigen-presenting dendritic cells alone or in combination with T cell enhancing agent(s). In an alternative approach, the methods of the invention include generating tumor-specific cytotoxic T cell ex vivo using the cultured dendritic cells and administering the generated tumor-specific cytotoxic T cells to the tumor-bearing subject. A T cell enhancing agent may be administered to the tumor bearing subject prior to obtaining T cells; alternatively or additionally, a T cell enhancing agent may be administered to the subject in conjunction with ex vivo-generated tumor-specific T cells.

The methods of the present invention further include administering a chemoattractant to attract mobilized dendritic cells and/or T cell, NK cells or other immune cells to a tumor site or another site (i.e., attracting antigen-carrying DC to a T cell-rich lymph node).

Combination immunotherapy methods described herein are useful in treating individuals suffering from immunosuppression that can occur in individuals who have received chemotherapy or radiation therapy or have cancerous cells, since many cancers have immunosuppressive effects. The immunotherapy methods of the invention stimulate an anti-tumor response and facilitate recovery of the immune system from the side effects of anti-tumor therapy.

Many DC mobilization factors enhance the population of bone marrow progenitor cells in the tumor-bearing subject. If desired, the inventive methods may be used as part of an immunization regimen to generate an effective immune response against a desired antigen in the tumor-bearing subject.

The inventive methods may be used to generate or regenerate an immune response in the tumor-bearing subject ex vivo by: (a) administering a therapeutically effective amount of a DC mobilization factor to the subject; (b) obtaining dendritic cells from the individual; (c) culturing the dendritic cells ex vivo; and (d) administering the dendritic cells to the individual at a time when anti-tumor therapy will not adversely affect the dendritic cells that are being administered.

In yet another aspect of the instant ex vivo therapy, the dendritic cells are treated with an antigen against which it is desired to generate an immune response in a manner similar to that described above for tumor antigen. Thus, the dendritic cells may also be caused to secrete certain desirable immunologically active agents; they may be administered alone or in combination with agents that enhance a cytotoxic T lymphocyte or helper cell response against the antigen, or a T cell growth factor to stimulate proliferation of T cells. Alternatively, the dendritic cells may be used to generate antigen-specific cytotoxic T cells or helper cells ex vivo, which are then administered to the tumor-bearing subject. These and other aspects of the invention will be apparent to one of ordinary skill in the art.

The present invention will also be useful in facilitating recovery of tumor-bearing individuals from immunosuppression that occurs as a result of anti-tumor therapy or as an effect of the tumor itself. An agent that increases the number of DC may be administered, and the DC obtained and preserved for subsequent re-administration to the individual. The DC may be treated ex vivo to allow them to more effectively present antigen to other immune cells; moreover, ex vivo techniques can also be applied to obtain antigen-specific effector cells such as cytotoxic T cells specific for a particular pathogenic or opportunistic organism.

Tumor-bearing subjects may also be treated with the inventive combination therapy after treatment that induces immunosuppression is completed, to reduce the amount of time that the tumor-bearing subject's immune response is diminished as a result of the immunosuppression-inducing treatment. Such combination therapy will reduce the risk that the individual will succumb to an infectious disease as a result of the immunosuppression-inducing treatment.

### BRIEF DESCRIPTION OF THE DRAWINGS

FIG. 1 is a flowchart depicting various steps in the inventive method(s). Those steps that must be performed in vivo are listed on the left side of the flow chart, while those that may be performed ex vivo are shown on the right side. While the steps are shown in the general order in which they would usually be performed, those of ordinary skill in the art are able to optimize the order and/or timing of the steps, as well as the dosages and routes of administration, by routine experimentation. Thus, for example, a tumor killing agent can be administered by any means disclosed herein; the optimal time to administer a dendritic cell (DC) maturation agent and/or cultured DC (either immature or activated, mature DC) will depend on the nature of the tumor killing agent and its effects, if any, on the DC. Similarly, as described in detail herein, when preparing mature, activated, antigen-carrying DC ex vivo, those of ordinary skill in the art will adjust the steps performed ex vivo to optimize activation and antigen presentation ability (i.e., generally, with peptide antigens, the DC are contacted with the peptide after maturation, whereas with larger antigens that require processing, the DC are usually contacted with the antigen and allowed to process it prior to maturation). Moreover, the skilled artisan can utilize chemoattraction to enhance trafficking of cells to a specific site by localized administration (achieved by any method described herein) of a chemokine or chemokine-inducing agent, for example, administering a chemokine (or chemokine inducer) that attracts DC intratumorally to increase the numbers of DC that take up tumor antigen, or administering a chemokine (or chemokine inducer) into a lymphnode to facilitate trafficking of antigen-carrying DC to a T cell-rich area. Additionally, an agent that enhances the numbers of circulating T cells can be administered to the tumor-bearing subject prior to obtaining T cells for ex vivo culture. The same agent (or another T cell enhancing agent) may be administered when expanded T cells are administered to the subject.

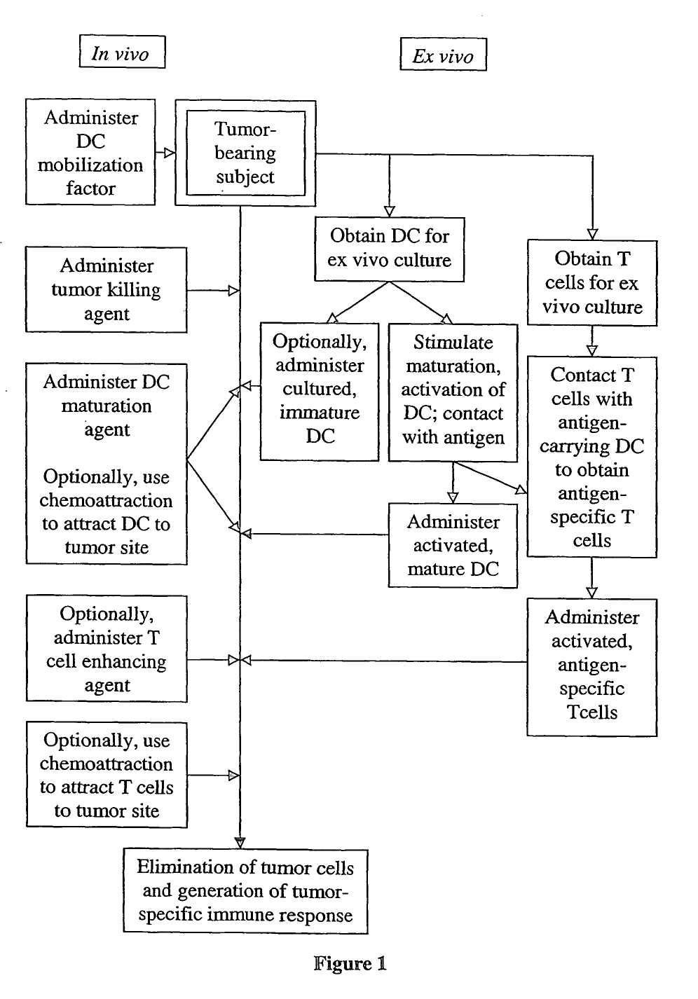

FIG. 2 presents the nucleotide and amino acid sequence of human granulocyte-macrophage colony stimulating factor.

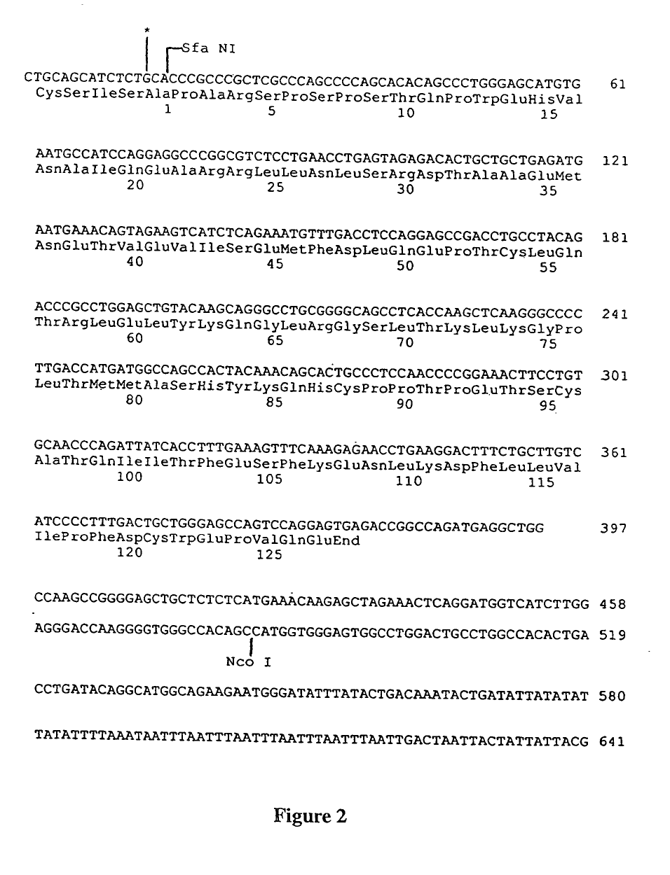

### DETAILED DESCRIPTION OF THE INVENTION

Advantageously, the methods of the present invention provide more highly available tumor antigen to sites near dying tumor cells or at sites draining dying tumor cells. The methods additionally increase dendritic cell (DC) populations for activation and maturation and enhance their ability to process and present tumor antigens to T cells. When treated according to the inventive methods, these tumor antigen-bearing DC induce a potent memory or primary T lymphocyte response specific to the tumor. T cell growth factors (either endogenously provided by activated DC or exogenously added) will further expand the tumor-specific CD4+ and CD8+ T cell population, which then facilitates the eradication of the remaining tumor burden.

The methods of the present invention include the use of combinations of agents in immune-based tumor therapies. Combinations of agents include separate, sequential or simultaneous administration of the agents. Agents suitable for use in the present invention include DC mobilization factors; tumor cell apoptotic agent and/or necrotic agents (tumor killing agents); DC maturation agents; T cell enhancing agents; and chemoattractants. The methods described herein include in vivo steps that encompass administering these agents directly to an individual and/or combinations of in vivo and ex vitro steps the involve contacting cells in in vitro manipulations.

In one embodiment, the present invention provides methods for treating tumor bearing individuals by administering to the individual: at least one DC mobilization factor; at least one tumor killing agent; and at least one DC maturation agent. In another embodiment, the inventive methods further include administration of at least one T cell enhancing agent to the individual. An additional embodiment further includes administration of tumor antigen(s) to the individual.

DC mobilization factors act to increase the number of DC or increase DC populations. Suitable dendritic cell mobilization factors (or agents) include, but are not limited to, Flt3L, granulocyte-macrophage colony stimulating factor (GM-CSF), granulocyte colony stimulating factor (G-CSF), CD40L and Interleukin-15 (IL-15). Different DC mobilization factors mobilize distinct subsets of DC in humans. Flt3L increases both CD11c+ and CD11c-IL-3R+ subsets; the former subset is increased between 40- and 50-fold and the latter is increased between 10- and 15-fold (Pulendran et al., J. Immunol. 165:566, 2000; Maraskovsky et al., Blood 96:878, 2000). In contrast, G-CSF increases only the CD11c-subset, and that by about 7-fold (Pulendran et al., supra). Because the two subsets of DC elicit different cytokine profiles in CD4+ T cells, different DC mobilization factors may be used to preferentially enhance one type of immune response over another (i.e., T_{H}1-like response versus T_{H}2-like response).

Flt3L refers to polypeptides that bind the cell-surface tyrosine kinase receptor Flt3, and regulate the growth and differentiation of progenitor and stem cells thereby. U.S. Pat. No. 5,554,512, issued Sep. 10, 1996 (herein incorporated by reference), describes the isolation of a cDNA encoding Flt3L, and the use of this molecule in peripheral stem cell transplantation procedures. Various forms of Flt3L are described therein, including both human and murine Flt3L, fusion proteins and muteins. Preferred Flt3L polypeptides comprise amino acids 28 through 160, amino acids 28 through 182, or amino acids 28 through 235 of human Flt3L (SEQ ID NO:1), and fragments thereof. Particularly preferred Flt3L polypeptides comprise amino acids 28 through 179 or amino acids 26 through 179 of SEQ ID NO:1)

Other Flt3L related dendritic cell mobilization agents suitable for use in the present invention include those agents that bind Flt3 and transduce a signal. Such Flt3 binding proteins encompass agonistic antibodies that include monoclonal antibodies and humanized antibodies, and recombinantly-prepared agents that have at least one suitable antigen binding domain and are derived from agonistic antibodies that transduce Flt3 signaling.

GM-CSF is a lymphokine that induces the proliferation and differentiation of precursor cells into granulocytes and macrophages. U.S. Pat. No. 5,162,111, issued Nov. 10, 1992, discloses the nucleotide and amino acid sequence of both human and murine GM-CSF, and describes the use of this lymphokine in treating bacterial diseases. Other forms of GM-CSF will also be useful in the instant invention, including fusion proteins comprising GM-CSF and Interleukin-3 (described in U.S. Pat. No. 5,108,910, issued Apr. 28, 1992), muteins of GM-CSF (disclosed in U.S. Pat. No. 5,391,485, issued Feb. 21, 1995), and prolonged-release compositions comprising GM-CSF (described in U.S. Pat. No. 5,942,253, issued Aug. 24, 1999). The relevant disclosures of the above-referenced patents are specifically incorporated herein.

IL-15 is a secreted cytokine that is produced as a precursor protein and cleaved to its active form. Mature IL-15 is capable of signaling the proliferation and/or differentiation of precursor or mature T-cells, and so can be used (in vivo or ex vivo) to regulate a T cell immune response. IL-15, which has been referred to as Epithelium-derived T-Cell Factor is described in U.S. Pat. No. 5,574,138, issued Nov. 12, 1996 (incorporated herein by reference). Preferred forms of IL-15 comprise mature IL-15 polypeptides (amino acids 49 through 162 of the non-cleaved precursor protein; SEQ ID NO:2).

Tumor killing agents include both apoptosis-inducing agents and necrosis-inducing agents, for example, radiation therapy, chemotherapy, ultrasound, photodynamic therapy, exposure to heat or very cold temperatures, antibody therapy, infection with viruses, transduction with viral vectors encoding selected proteins, and various members of the Tumor Necrosis Factor (TNF) superfamily (including TNF, Lymphotoxins alpha and beta, CD40L, and TNF-related apoptosis-inducing or TRAIL). Radiation and/or chemotherapy are among the tools used by oncologists to treat various forms of cancer or precancerous conditions. The preferable mode of treatment will depend on the specific type of cancer being treated, the stage of the disease, and the condition of the patient, among other factors. Those of skill in the art of treating cancer and precancerous conditions are aware of varying treatment regimens that may be used, and will apply their skills to determine a preferred regimen based on their knowledge of each individual situation. Several texts are useful to assist the skilled artisan in selecting a regimen, including Cancer: Principles and Practice of Oncology, 5th Edition (DeVita, Hellman and Rosenberg, eds; Lippincott-Raven Publishers, 1997), and Principles and Practice of Radiation Oncology, 3rd edition (Perez and Brady, eds.; Lippincott Williams and Wilkins Publishers, 1997).

Various computer and Internet-based resources are available to assist in determining apoptotic agents and apoptotic modes. An exemplary web site is that maintained by the National Cancer Institute of the National Institutes of Health of the U.S. (http://cancernet.nci.nih.gov/). The website contains information about various types of cancer, treatment options, various clinical trials that are ongoing, risk factors in cancer, and other helpful resources.

A number of antibody therapies are suitable tumor killing therapies in the practice of the present invention. Rituxan® (Rituximab; IDEC Pharmaceuticals, San Diego, and Genentech, Inc, San Francisco, Calif.) is a chimeric monoclonal antibody against the cell-surface marker CD20 that mediates complement-dependent cell lysis and antibody-dependent cellular cytotoxicity of CD20-expressing cells. It has also been shown to sensitize chemoresistant human lymphoma cell lines and to induce apoptosis. Rituxan® has been shown to have clinically significant effect in treatment of CD20-positive lymphomas (McLaughlin et al., J. Clin. Oncol. 16:2825; 1998).

Herceptin® (Trastuzumab; Genentech, Inc., South San Francisco, Calif.) is a humanized monoclonal immunoglobulin G1 kappa antibody that binds with high affinity and specificity to the extracellular domain of human epidermal growth factor receptor 2 (HER2). Preclinical studies have shown that administration of Herceptin” alone or in combination with paclitaxel or carboplatin significantly inhibits the growth of breast tumor-derived cell lines that overexpress the HER2 gene product. A description of Herceptin® is given in U.S. Pat. No. 6,054,297, issued Apr. 25, 2000, the disclosure of which is incorporated by reference herein.

IMC-C225 is another antibody that blocks a growth factor receptor found on a variety of tumor cells. IMC-C225 is a chimeric antibody, developed and produced by ImClone Systems Incorporated (New York, N.Y.) and is described by Overholser et al. (Cancer 89:74; 2000). The antibody acts by blocking the growth factor receptor and preventing tumor cells from evading cell death signals.

Another useful antibody is ABX-EGF, a human IgG₂ monoclonal antibody generated in transgenic mice, that binds human epidermal growth factor receptor (EGFr) with high affinity (Yang et al., Crit Rev Oncol Hematol 38:17, 2001). ABX-EGF blocks the binding of both EGF and transforming growth factor-alpha (TGF-alpha) to EGFr-expressing human carcinoma cell lines, and inhibits EGF-dependent tumor cell activation. Because it is a fully human antibody, ABX-EGF will likely exhibit a long serum half-life and minimal immunogenicity in human patients.

Tumor killing agents include polypeptides that induce apoptosis of certain target cells including, but not limited to, TRAIL. TRAIL induces apoptosis of cancer cells and virally-infected cells. The cloning and characterization of TRAIL is described in U.S. Pat. No. 5,763,223, issued Jun. 9, 1998. As disclosed therein, TRAIL comprises an N-terminal cytoplasmic domain, a transmembrane region and an extracellular domain. Soluble forms of TRAIL that are useful in the present invention include the extracellular domain of TRAIL or a fragment of the extracellular domain that retains the ability to bind to target cells and induce apoptosis. A preferred form of soluble TRAIL comprises amino acids 95 through 281 of human TRAIL (SEQ ID NO:5) as disclosed in U.S. Pat. No. 5,763,223.

Oligomeric forms of TRAIL are also useful; preferred forms comprise the extracellular domain of TRAIL fused to a peptide that facilitates trimerization. Peptides derived from naturally occurring trimeric proteins or synthetic peptides that promote oligomerization may be employed. Particularly useful peptides are those referred to as leucine zippers (zipper domains or leucine zipper moieties). In particular embodiments, leucine residues in a leucine zipper are replaced by isoleucine residues. Such peptides comprising isoleucine may be referred to as isoleucine zippers, but are encompassed by the term “leucine zippers” as employed herein.

One example is a leucine zipper derived from lung surfactant protein D (SPD), as described in Hoppe et al. (FEBS Letters 344:191, 1994) and in U.S. Pat. No. 5,716,805, comprising amino acids Pro Asp Val Ala Ser Leu Arg Gln Gln Val Glu Ala Leu Gln Gly Gin Val Gln His Leu Gln Ala Ala Phe Ser Gln. Another example of a leucine zipper that promotes trimerization is the zipper peptide shown in SEQ ID NO:4. In an alternative embodiment, the peptide lacks the N-terminal Arg residue. In another embodiment, an N-terminal Asp residue is added. Yet another example of a suitable leucine zipper peptide comprises the amino acid sequence Ser Leu Ala Ser Leu Arg Gln Gln Leu Glu Ala Leu Gln Gly Gln Leu Gln His Leu Gln Ala Ala Leu Ser Gln Leu Gly Glu. In an alternative peptide, the leucine residues in the foregoing sequence are replaced with isoleucine. Fragments of the foregoing zipper peptides that retain the property of promoting oligomerization may be employed as well. Examples of such fragments include, but are not limited to, peptides lacking one or two of the N-terminal or C-terminal residues presented in the foregoing amino acid sequences.

Suitable DC maturation agents useful in the practice of the invention include CD40L and agonists of CD40 signaling, RANKL, TNF, IL-1, CpG-rich DNA sequences (ISS, or immunostimulatory sequences), lipopolysaccharide (LPS), and monocyte-conditioned medium (Reddy et al., Blood 90:3640;1997). These agents act on DC by enhancing their capabilities to stimulate an effective, specific, anti-tumor cytoxic response. Thus, for example, administering CD40L or contacting DC with CD40L causes the ligation of CD40 expressed on DC, which in turn stimulates an increase in the numbers of MHC molecules on the surface of DC. This increases the antigen-presenting capacity of the DC. Administering maturation agents or contacting DC with maturation agents also enhances the secretion of various immunomodulatory cytokines (for example, IL-12) which can act to augment the anti-tumor response. DC may also be contacted with agents that stimulate secretion of cytokines that indicate that the DC are activated (DC activation factors). Thus, for example, DC may be contacted with CD40L and IFN-γ (simultaneously, sequentially or separately) to stimulate maturation and activation of DC.

CD40L polypeptides that are capable of binding CD40, and transducing a signal thereby, are useful in the present invention. cDNAs encoding CD40L are described in U.S. Pat. Nos. 5,961,974, 5,962,406 and 5,981,724 (hereinafter, the Armitage patents). Forms of CD40L that are particularly useful maturation agents include the extracellular portion of CD40L and fragments of the extracellular portion that bind CD40 and transduce a signal. In particular, polypeptides that include amino acids 47-261 of SEQ ID NO:3, polypeptides that include amino acids 113-261 of SEQ ID NO:3, polypeptides that include amino acids 51-261 of SEQ ID NO:3 and oligomeric forms of these polypeptides, as disclosed in the Armitage patents, can be used in the present invention. A preferred CD40L is one in which the cysteine amino acid 194 of human CD40L is substituted with tryptophan. A most preferred form of CD40L is a soluble CD40L fusion protein referred to as trimeric CD40L in the Armitage patents. Trimeric CD40L comprises a fragment of the extracellular domain of CD40L fused to a zipper domain that facilitates trimerization (SEQ ID NO:4).

Additional suitable dendritic cell maturation agents include compounds that bind CD40 and transduce a signal. Amongst these are agonistic antibodies to CD40 such as monoclonal antibody HuCD40-M2 (ATCC HB 11459) as well as humanized antibodies or other, recombinantly-derived molecules comprising an antigen binding domain derived from antibody HuCD40M2.

RANKL, like CD40L, is a Type 2 transmembrane protein with an intracellular domain of less than about 50 amino acids, a transmembrane domain and an extracellular domain of from about 240 to 250 amino acids (SEQ ID NO:6). RANKL is described in U.S. Ser. No. 08/995,659, filed Dec. 22, 1997 (PCT/US97/23775). Similar to other members of the TNF family to which it belongs, RANKL has a spacer region between the transmembrane domain and the receptor binding domain that is not necessary for receptor binding. Accordingly, soluble forms of RANKL can comprise the entire extracellular domain or fragments thereof that include the receptor binding region.

Similarly to CD40L, other compounds that bind RANK and transduce a signal are useful maturation agents and include agonistic antibodies to RANK as well as humanized antibodies or other, recombinantly-derived molecules comprising an antigen binding domain derived from antibody that binds RANK. Several other members of the TNF superfamily will also have use in various aspects of the instant invention. These include lymphotoxins alpha and beta, Fas ligand, CD27 ligand, CD30 ligand, CD40 ligand, 4-1BB ligand, OX40 ligand, TRAIL and RANKL.

DC can also be grown ex vivo after mobilization with Flt3L, GM-CSF, granulocyte colony stimulating factor (G-CSF), cyclophosphamide or other agents known to mobilize CD34+ cells. The DC so obtained can be cultured using agents such as Flt3L, GM-CSF, Interleukin-15 (IL-15), CD40 Ligand (CD40L) or the ligand for receptor activator of NF-kappaB (RANKL). Alternatively, DC can be generated from peripheral blood mononuclear cells (PBMC) using GM-CSF and Interleukin-4 (IL-4). Cultured DC can further be treated ex vivo to stimulate maturation and/or activation as described above. The DC generated ex vivo by these methods may be administered locally into a tumor, systemically into the bloodstream or into draining lymph nodes.

TNF is a dendritic cell maturation agent that also plays a central role in inflammatory and immune defenses, and is involved in several pathogenic processes, including cachexia, septic shock and autoimmunity. Its potent effects on cells of the immune system render it useful in vitro (for example, in ex vivo generation, expansion and/or activation of cells, and/or maturation of DC). Moreover, various techniques can be used to minimize systemic effects, for example, use in gene therapy or local administration in or near the site of a tumor, as discussed herein.

Lipopolysaccharide (LPS), another dendritic cell maturation agent, is a component of the cell wall of Gram-negative bacteria. LPS consists of a lipid core (lipid A) and an attached polysaccharide moiety; the lipid A (along with some associated polysaccharides) is thought to be responsible for most of the toxic effects of Gram-negative bacteremia, including toxic shock syndrome (septic shock or endotoxemia). LPS may be used ex vivo to generate mature DC; alternatively, various techniques described herein can be applied to allow for localized administration of LPS to a tumor-bearing subject.

Additional suitable dendritic cell maturation agents include those agents that are also suitable T-cell enhancing agents. Such agents include Interleukins 2, 15, 7 and 12, (IL-2, IL-15, IL-7, and IL-12, respectively) and interferons-gamma and -alpha (IFN-γ and IFN-α), and OX40 and 4-1BB agonists. These agents, and many others that have utility in the present combination therapy method, are described in The Cytokine Handbook (third edition; edited by Angus Thompson; Academic Press 1998).

First identified as a T cell growth factor, Interleukin-2 (IL-2) is also known to affect B cells, natural killer (NK) cells, lymphokine-activated killer (LAK) cells, monocytes, macrophages and oligodendrocytes. U.S. Pat. No. 6,060,068, issued May 9, 1000, describes IL-2 and its use as a vaccine adjuvant. IL-2 in gene therapy is described in U.S. Pat. No. 6,066,624, issued May 23, 2000. The use of IL-2 in conjunction with heat shock protein/antigenic peptide complexes for the prevention and treatment of neoplastic disease is described in U.S. Pat. No. 6,017,540, issued Jan. 25, 2000.

Another dendritic cell maturation agent and T-cell enhancing agent, Interleukin-7 (IL-7) is a cytokine of about 25 KDa that is secreted by both immune and non-immune cells, and is involved in the development of the immune systems and the generation of a cellular immune response. U.S. Pat. No. 5,328,988, issued Jul. 12, 1994, describes the identification and isolation of human IL-7. Because IL-7 enhances the immune effector cell functions of T lymphocytes, and is useful in the practice of the present invention as a T-cell enhancing agent in its ability to augment a CTL response. IL-7 also acts as a growth factor and has been used to stimulate the growth of immune cells after bone marrow transplantation or high-dose chemotherapy. Accordingly, IL-7 is also useful in the instant invention as an agent that mobilizes or stimulates the growth of immune cells prior to induction of tumor cell death.

Interleukin-12 (IL-12) is a heterodimeric protein that has a heavy chain (p40) that bears structural resemblance to the Interleukin-6 (IL-6) receptor and the G-CSF receptor, and a light chain (p35) that resembles IL-6 and G-CSF. Because of its ability to promote the preferential development of a T_{H}1 immune response, IL-12 has been used in the infectious disease setting as well as in tumor models. IL-12 is a useful T-cell enhancing agent and provides enhanced anti-tumor CTL activity in methods of the present invention. Administering IL-12 can induce tumor cell apoptosis, and thus IL-12 is useful in instant invention as an apoptotic agent. Additionally, IL-12 DNAs may be used in in vitro methods, for example by transducing tumor cells or dendritic cells to express IL-12, then administering the cell intratumorally.

Interferons fall into two categories referred to as Type I interferons (IFN-α, IFN-ω, IFN-β and IFN-τ) which exhibit structural homology and are believed to be derived from the same ancestral gene, and Type II Interferon (IFN-γ) which does not exhibit homology with the other interferons, but shares some biological activities. Both types of interferons enhance the expression of MHC molecules, which augment the cytolytic activity of T cells, thus making interferons useful T-cell enhancing agents. Interferons also activate natural killer (NK) cells, and macrophages, both of which become more effective at killing tumor cells. Moreover, some tumor cells are directly affected by interferons, which may slow down their growth or proliferation. Numerous patents describe the production and use of various interferons. For example, U.S. Pat. No. 5,540,923 describes methods for isolating both Type I and Type II interferons and U.S. Pat. Nos. 5,376,567 and 4,889,803 relate to the recombinant expression of IFN-γ. A form of IFN-γ 1b known as Actimmune™ is manufactured by InterMune, Palo Alto, Calif. Low-doses of IFN-α have been used in treating chronic myeloid leukemia (Schofield et al., Ann. Intern. Med. 121:736; 1994) and other forms of cancer. A recombinant from of IFN-α, Introna®, is marketed by Schering-Plough for various anti-viral and anti-cancer indications.

Other agents that act on the various members of the TNF receptor superfamily of proteins will also have utility herein. Exemplary agents include agonistic antibodies, including humanized or single chain versions thereof. For example, Melero et al. have shown that monoclonal antibodies to 4-1BB can lead to the eradication of large, poorly immunogenic tumors in mice (Nature Med. 3:682; 1997). According to Melero et al., agonistic 4-1BB antibodies augment tumor-specific CTL activity. Accordingly, such antibodies (or 4-1BB ligands) may have use in the inventive method for upregulating CTL activity; they may also function to increase the amount of tumor antigen available by causing tumor cell death. U.S. Pat. No. 5,674,704, issued Oct. 7, 1997, discloses a ligand for 4-1BB that comprises a cytoplasmic domain, a transmembrane region and an extracellular domain. A soluble form of 4-1BB ligand comprising the extracellular domain is also disclosed; additional, multimeric forms are prepared by adding a multimer-forming peptide (such as an Fc molecule or a zipper peptide) to the extracellular domain. A particularly useful agonistic monoclonal antibody is 4-1BBm6 (deposited at the American Type Tissue Collection in Manassas, Va. on ______ and given accession number ______). Other forms of antibodies that bind the same epitope as 4-1BBm6 will also be useful, including humanized forms of murine antibodies, single chain antibodies, and monoclonal antibodies that are generated in transgenic mice that exhibit human antibody genes and therefor make human antibodies to antigens.

Similarly, agonists of OX40 (molecules that bind OX40 and transduce a signal thereby, including agonistic antibodies and OX40 ligand) promote a CD8+ T cell response that can lead to the rejection of tumors. U.S. Pat. No. 5,457,035, issued Oct. 10, 1995, discloses a ligand for OX40; Miura et al. (Mol. Cell Biol. 11:1313; 1991) disclose a human homolog of murine OX40L which they refer to as gp34. Like other members of the TNF superfamily, OX40L is a type II transmembrane protein; soluble forms of OX40L are made from the extracellular domain. Multimeric forms of OX40L are prepared using standard recombinant DNA techniques to append a multimer-forming peptide such as an immunoglobulin Fc or an oligomerizing zipper to DNA encoding OX40L. A preferred agonistic monoclonal antibody is Ox40 m5 (deposited at the American Type Tissue Collection in Manassas, Va. on ______ and given accession number ______). Other forms of antibodies that bind the same epitope as Ox40 m5 will also be useful, including humanized forms of murine antibodies, single chain antibodies, and monoclonal antibodies that are generated in transgenic mice that exhibit human antibody genes and therefor make human antibodies to antigens.

Those of skill in the art are also aware of a number of other factors that influence T cells, including Transforming Growth Factor-β (TGF-β). This cytokine can enhance the growth of immature lymphocytes, inhibit the apoptosis of T cells, and has a potent immunosuppressive effect on lymphocytes. Thus, TGF-β or inhibitors thereof (such as antibodies that bind TGF-β and prevent binding to cell-associated TGF-β receptor, soluble forms of TGF-β receptors, or other molecules that interfere with the ability of TGF-β to bind its receptor or transduce a signal thereby) will also be useful in the instant invention. The skilled artisan will be able to select appropriate forms to use, depending on the desired effects, by the application of routine experimentation.

Other molecules are also known to be crucial in the development of an immune response, and appear to preferentially enhance an immune response that is T_{H}2-like (that is, dominated by antibody-producing cells with little or no generation of cytotoxic T cells), including Interleukins 4, 5 and 10. Antagonists of these molecules will be useful in preventing or decreasing a T_{H}2-like immune response; in combination with the other aspects of the present invention, such antagonists facilitate the manipulation of an immune response toward a T_{H}1-like response, which may be more effective at eliminating tumor cells in an individual. Antagonists include antibodies that bind one of these molecules and prevent binding to cell-associated receptors therefor, soluble forms of receptors, or other molecules that interfere with the ability of the molecule to bind its receptor or transduce a signal thereby. U.S. Pat. No. 5,599,905, issued Feb. 4, 1997, discloses useful forms of soluble IL4 receptor.

Chemokines are small, basic proteins that exhibit chemotactic activity for various types of immune system cells. The members of this family of proteins can be divided into roughly four groups based on the formation of disulphide bonds between cysteine residues and the presence or absence of intervening amino acids between the cysteine residues, which correlate approximately with function. Thus, members of the CXC subgroup exhibit an intervening amino acid between the first two hallmark cysteine residues, and tend to mainly attract and activate neutrophils. CC chemokines do not have an intervening amino acid, and exhibit chemotactic activity for dendritic cells, lymphocytes and mononuclear cells. The third subclass of chemokines is the C family, which lacks two of the four cysteines; it is represented by lymphotactin, a lymphoid-specific attractant that has been shown to attract NK and CD4 T cells to tumor sites. A fourth type of chemokine with three intervening amino acids (CX3C) has also been identified; the representative molecule of this subfamily, fractalkine, may be involved in leukocyte adhesion and extravasation.

Accordingly, chemokines will find use in the instant invention to attract particular types of cells to the tumor site. For example, a CC chemokine such as one of MCPs 1-5, MIP-1 alpha or beta, RANTES or eotaxin, may be given locally at the site of the tumor by any of the techniques known in the art and discussed herein (i.e., by intra tumoral injection of the protein or DNA encoding it, or through use of a gene therapy technique to induce secretion of the chemokine by cells at the site of the tumor), to attract mobilized dendritic cells to the site. The chemokine used can be selected, depending on the type of cell to be attracted, by the application of routine experimentation.

Additional useful agents are disclosed in U.S. Ser. No. 60/249,524, filed Nov. 17, 2000, the disclosure of which is incorporated by reference herein. In particular, the chemokines MIP-3alpha, MIP-3beta, MIP-5, MDC, SDF-1, MCP-3, MCP-4, RANTES, TECK, and SDF-1 are useful chemokines that act as dendritic cell localization factors. Moreover, cytokines such as IL-1, TNF-alpha and IL-10 are also capable of acting as localization factors. Compounds that bind to and activate one or more members of the somatostatin cell surface receptors SSTR1, SSTR2, SSTR3, SSTR4 and SSTR5 or homologs or orthologs thereof will also be useful in the inventive methods. These include the naturally occurring ligands for the somatostatin receptors, including somatostatin and cortistatin, and somatostatin peptides SST-14, SST-28 and cortistatin peptides CST-17 and CST-29. Other known peptide agonists of SSTRs include ocreotide, lanreotide, vapreotide, seglitide, BIM23268, NC8-12, B1M23197, CD275 and other found to have high affinity for SSTRs. Derivatives, analogs and mimetics of any of these compounds will also be useful in the present invention.

It is understood by those of skill in the art that the various agents and/or factors disclosed herein act by binding to cell surface receptors and transducing a signal to the cell thereby. It is also understood that other agents can also exhibit these characteristics (i.e., agonistic antibodies to a given receptor). Accordingly, the inventive methods encompass the use of other molecules that mimic the signaling to cells that occurs with the factors that are specifically disclosed above. Such molecules include agonistic monoclonal antibodies and recombinant proteins derived therefrom as well as ligand mimetics isolated by screening small molecule libraries or through rational drug design.

Those of skill in the art also understand that useful recombinant proteins can be expressed in forms that differ from the corresponding native protein. For example, certain members of the TNF family of proteins are believed to exist in trimeric form (Beutler and Huffel, Science 264:667, 1994; Banner et al., Cell 73:431, 1993). Preferred forms of TNF family members may comprise a peptide that facilitates trimerization (or other multimerization) as described herein for CD40L or TRAIL.

Administration of Agents that Stimulate Tumor Cell Death

Various means of inducing tumor cell death are known in the art; the exact method of administration will depend on the type of cancer being treated, the stage of the cancer and the health of the patient, among other factors. Those of skill in the art will be able to select appropriate methods of administration based on these factors. Generally, if the factor is a chemotherapeutic agent (or combination thereof), or a biologic agent, it will be administered in the form of a pharmaceutical composition comprising purified compound in conjunction with physiologically acceptable carriers, excipients or diluents. Such carriers are nontoxic to subjects at the dosages and concentrations employed.

Ordinarily, the preparation of such compositions entails combining a compound with buffers, antioxidants such as ascorbic acid, low molecular weight (less than about 10 residues) polypeptides, proteins, amino acids, carbohydrates including glucose, sucrose or dextrans, chelating agents such as EDTA, glutathione and other stabilizers and excipients. Neutral buffered saline or saline mixed with conspecific serum albumin are exemplary appropriate diluents. Moreover, various forms of controlled release technology may be employed; U.S. Pat. No. 5,942,253 discloses prolonged-release compositions comprising GM-CSF. Such compositions and others that can be prepared by those of ordinary skill in the art (for example, the use of hydrogels as disclosed herein) will also be useful in the instant invention. The particular therapeutic effective amount employed is not critical to the present invention, and will vary depending upon the particular factor selected, the disease or condition to be treated, as well as the age, weight and sex of the subject.

Chemotherapeutic agents act on cancer cells to inhibit their growth; many of the side effects of chemotherapy are due to the damage that these agent cause to normal, rapidly dividing cells. Patients afflicted with cancer may be treated by chemotherapy alone, or in combination with other anti-cancer treatments. Numerous chemotherapeutic agents are known; some are effective against numerous types of tumors and are used to treat many different kinds of cancer, while others are most effective for just one or two types of cancer. Chemotherapeutic agents may be given intravenously, orally, by injection, or applied to the skin. Accordingly, whether chemotherapy is used and which agent or combination thereof should be given depends on the type of cancer, location of the tumor, and the health of the patient, among other factors.

Another form of treatment that has been used to cause tumor cell death is cryosurgery (or cryotherapy), in which extreme cold is applied to cancer cells, causing cell death. Cryosurgery has been most frequently used to treat skin tumors (or other external tumors), by applying liquid nitrogen directly to the tumor. However, techniques to allow the use of extreme cold in treating internal tumors have been developed. For example, liquid nitrogen may be circulated through a cryoprobe, using ultrasound to monitor and direct application of the liquid nitrogen to tumor cells while minimizing damage to the surrounding normal cells. Cryosurgery has been used, or is being investigated, for treating various types of skin cancer, retinoblastoma, prostate cancer, liver cancer, for tumors of the bone, brain and spinal cord, and tumors that form in the esophagus; it is also used precancerous conditions such as actinic keratosis and cervical intraepithelial neoplasia. In addition, cryotherapy has been used successfully in the treatment of warts (which may in some instances to cancerous or precancerous conditions) and molluscum contagiousum; use of the combination therapy disclosed herein may also prove beneficial in such conditions.

High temperatures (hyperthermia) have also been used in efforts to eradicate cancer cells, usually in combination with other types of therapy. In local hyperthermia, heat is applied to a tumor, using high-frequency waves aimed at a tumor from a device outside the body, sterile probes (thin, heated wires or hollow tubes filled with warm water), implanted microwave antennae; or radiofrequency electrodes. Limbs or organs may also be heated in a process referred to as regional hyperthermia. In this technique, magnets or devices that produce high energy are placed over the region to be heated, or the limb or organ is heated by perfusion (removing, some of the patient's blood, heating it, and returning it to the organ or limb) Whole-body heating may be used to treat metastatic cancer through the use of warm-water blankets, hot wax, inductive, or thermal chambers.

Radiation therapy utilizes ionizing radiation to damage the genetic material of cells; both normal and cancerous cells can be damaged, but normal cells retain the ability to repair the damage whereas this ability is diminished in cancer cells. Localized solid tumors are often treated using radiation therapy; leukemias and lymphomas may also be treated with radiation therapy. Several types of ionizing radiation can be used, including X-rays and gamma rays. Radiotherapy can be applied using a machine to focus the radiation on the tumor, or by placing radioactive implants directly into the tumor or in a nearby body cavity. Moreover, radiolabeled antibodies can be used to target tumor cells. Scientists are also investigating other radiotherapy techniques, including intraoperative irradiation, and particle beam radiation, as well as the use of radiosensitizers (including heat) to make tumor cells more sensitive to radiation, or radioprotectants to protect normal cells.

Another type of cancer therapy, photodynamic therapy (PDT), utilizes light energy to kill cancer cells. In PDT, a photosensitizer is administered to the patient, who is subsequently exposed (usually only the affected body area) to light. Various modes of administering a photosensitizer are known in the art, and will be useful in the present invention. For example, the photosensitizer may be administered orally, topically, parenterally, or locally (i.e., directly into or near the tumor or precancerous area). The photosensitizers may also be delivered using vehicles such as phospholipid vesicles or oil emulsions. Use of lipid-based delivery vehicles may result in enhanced accumulation of the photosensitizer in neoplastic cells. Alternative methods of delivery also encompassed in the instant invention include the use of microspheres, or monoclonal antibodies or other proteins that specifically bind a protein (or proteins) located on the surface of neoplastic cells.

The particular photosensitizer employed is not crucial to the present invention. Examples of photosensitizers useful in the present invention include hematoporphyrins, uroporphyrins, phthalocyanines, purpurins, acridine dyes, bacteriochlorophylls, bacteriochlorins and others are disclose herein. A preferred photosensitizer employed is Photofrin® (QLT, Vancouver, Canada); additional examples are disclosed herein, and discussed in Dougherty et al. as well as various other resources disclosed herein. Further examples of photosensitizers are discussed in U.S. Ser. No. 09/799,785, filed Mar. 6, 2001, published as US Patent Application 20010022970. As is true for chemotherapeutic agents, the amount of photosensitizer administered will vary depending upon the particular photosensitizer employed, the age, weight and sex of the subject, and the mode of administration, as well as the type, size and location of the tumor.

Moreover, the wavelength of light to which the subject is exposed will vary depending upon the photosensitizer employed, and the location and depth of the tumor or precancerous cells. Generally, the subject will be exposed to light having a wavelength of about 600 to 900 nm, preferably about 600 to about 640 nm for Photofrin®. Several other photosensitizing agents have stronger absorbances at higher wavelengths, from about 650 to 850 nm, which can be beneficial for deeper tumors because longer wavelength light tends to penetrate further into tissue. Conversely, a wavelength of about 410 nm may give better results when shallow penetration is desired; such dosages also fall within the scope of this invention.

The dose of light to which the subject is exposed will vary depending upon the photosensitizer employed. Generally, the subject will be exposed to light dose of about 50 to 500 J/cm² of red light, for Photofrin”. Other sensitizers may be more efficient, and thereby require smaller fluences, typically about 10 J/cm². At higher fluences, hyperthermia may occur, which can enhance PDT; moreover, hyperthermia and PDT may act synergistically. Accordingly, the present invention encompasses are encompasses herein. Several different light sources are known in the art; any suitable light source capable of delivering an appropriate dosage of a selected wavelength may be used in the inventive methods.

The timing of light exposure will depend on the photosensitizer used, the nature and location of the tumor or precancerous cells, and the methods of administration. Typically, light exposure occurs at about one hour to four days after administration of the photosensitizer. Moreover, shorter time periods may be used, again depending on the photosensitizer, and the nature and location of the tumor. For example, light exposure after topical administration of a photosensitizer may occur as early as about ten minutes, or at about three hours after administration (see U.S. Pat. No. 6,011,563, which is incorporated by reference herein in its entirety).

Yet another method for inducing tumor cell death involves the application of gene therapy techniques. U.S. Pat. No. 6,066,624, issued May 23, 2000 describes a method of treating localized tumors by introducing a ‘suicide gene’ into tumor cells. In this technique, a recombinant adenoviral vector comprising a suicide gene is delivered into the tumor. The patient is then given a prodrug, which is acted upon by the protein encoded by the suicide gene, resulting in death of the tumor cell. Moreover, cytokine genes may also be introduced into tumor cells using such techniques; the cytokines may act to make the tumor more immunogenic. Viral vectors may also be used to deliver normal tumor suppressor genes or oncogene inhibitors into tumor cells. Thus, for example, tumors that express mutant forms of p53 can be transfected with a wild-type p53. leading to growth arrest of the tumor cells. CD148, a receptor-like protein tyrosine phosphatase, is a protein appears to be down regulated in some cancer cells (Autschbach et al., Tissue Antigens 54:485; 1999); introduction of CD148-encoding DNA into cancerous cells may lead to their growth arrest.

Localized administration may allow the use of tumor-killing agents that are not desirable for systemic use (for example, TNF, FasL, or very high doses of CD40L), and may be used to achieve higher concentrations of various agents at the site of the tumor than could safely be achieved using systemic administration. Various means may be used to achieve localized administration, including intratumoral injection of protein, use of gene therapy techniques to induce expression of recombinant protein in or near the tumor, and use of site-specific and/or controlled release technology. Moreover, it has been found that raw DNA, when injected into a mammal, is often taken up by cells and expressed. Accordingly, DNA encoding a desired factor may be injected into or near the site of a tumor, and, when taken up by nearby cells, will result in the localized expression of the factor encoded thereby.

One type of technology that may be useful for localized administration is that utilizing hydrogel materials to achieve sustained release of a desired factor or factors, for example, photopolymerizable hydrogels (Sawhney et al., Macromolecules 26:581; 1993). Similar hydrogels have been used to prevent postsurgical adhesion formation (Hill-West et al., Obstet. Gynecol. 83:59; 1994) and to prevent thrombosis and vessel narrowing following vascular injury (Hill-West et al., Proc. Natl. Acad. Sci. USA 91:5967; 1994). Proteins can be incorporated into such hydrogels to provide sustained, localized release of active agents (West and Hubbell, Reactive Polymers 25:139; 1995; Hill-West et al., J. Surg. Res. 58:759; 1995).

Accordingly, the various factors disclosed herein can also be incorporated into hydrogels, for application to tissues for which localized administration is desirable. For example, a hydrogel incorporating a tumor-killing agent, DC attractant, DC maturational factor, or CTL enhancing factor, or a combination of various such factors, can be applied to tissue after surgical removal or reduction of the tumor. Moreover, such hydrogel-based formulations may be administered by other methods that are known in the art, for example using a catheter to apply the hydrogel at a desired location in the vascular system, or by any other means by which intratumoral administration can be accomplished. Those of ordinary skill in the art will be able to formulate an appropriate hydrogel by applying standard pharmacokinetic studies, for example as discussed by West and Hubbell, supra.

Administration of Factors that Regulate an Anti-Tumor Response

The DC mobilization factors, DC maturation factors, DC attractant factors and T cell enhancing factors may be administered in a suitable diluent or carrier to a subject, preferably a human. Thus, for example, any one or all of these factors can be given by bolus injection, continuous infusion, sustained release from implants, or other suitable technique. Moreover, the factors can be administered locally (i.e., intratumoral administration), or by using gene therapy techniques. For example, tumor cells can be transfected with a gene encoding a CTL enhancing factor such as IL-2, IL-12, or IL-15. The transfected tumor cells are administered (for example, intratumorally) to the individual to provide a stronger and improved immune response to the antigen. Those of skill in the art will be able to perform routine experimentation using animal models or other modeling systems to determine preferable routes of administration and amounts of various factors to deliver (see, for example, the discussion in U.S. Pat. No. 6,017,540, issued Jan. 25, 2000, relating to dosage calculations and animal models).

Typically, a factor will be administered in the form of a pharmaceutical composition comprising purified compound in conjunction with physiologically acceptable carriers, excipients or diluents. Such carriers are nontoxic to subjects at the dosages and concentrations employed. Ordinarily, the preparation of such compositions entails combining a compound with buffers, antioxidants such as ascorbic acid, low molecular weight (less than about 10 residues) polypeptides, proteins, amino acids, carbohydrates including glucose, sucrose or dextrans, chelating agents such as EDTA, glutathione and other stabilizers and excipients. Neutral buffered saline or saline mixed with conspecific serum albumin are exemplary appropriate diluents.

The particular therapeutically effective amount employed is not critical to the present invention, and will vary depending upon the particular factor selected, the disease or condition to be treated, as well as the age, weight and sex of the subject. Additionally, the time at which a given factor is given will depend on the individual factor administered and its activity. Typically, a DC mobilization factor is given from ten to fifteen days prior to administration of the agent that induces tumor cell death, and may continue for five to ten days after administration of the tumor-killing agent. A DC maturation factor is given 24 to 48 hours after induction of tumor cell death; a T cell-enhancing agent is given at about the same time.

When the agent that causes tumor cell death is given over an extended time period (as in certain chemotherapy and radiation regimens), the effect of continuing therapy on the dendritic cells and T cells must be considered when designing a regimen for administration of the DC mobilization and maturation factors and T cell enhancing agents. For example, when the continuing therapy would result in killing of the mobilized and/or activated DC and/or CTL, DC maturational and T cell enhancing factors are not given until after the continuing therapy is completed, or at such a time point in the continuing tumor-killing regimen that there will be sufficient time for the anti-tumor immune response to mature to a stage in which the effector cells are less likely to be negatively affected by the continuing tumor-killing therapy.

Typical therapeutically effective dosages of various factors and typical intervals at which to administer them are shown in Table 1 below. Those of ordinary skill in the art are able to optimize dosages and routes of administration of these and other factors by the application of routine experimentation.

TABLE 1
Typical Therapeutic Dosages

| Factor | Dosage Range | | Administer at: |
|---|---|---|---|
| Flt3L | 25-100 | μg/Kg | 10 to 15 days prior to induction |
| | | | of tumor cell death through 5 to |
| | | | 10 days after induction of tumor |
| | | | cell death; daily or every other day; |
| | | | or via slow or controlled release |
| GM-CSF | 100-300 | μg/Kg | 10 to 15 days prior to induction of |
| | | | tumor cell death through 5 to 10 |
| | | | days after induction of tumor cell |
| | | | death; daily or every other day; or |
| | | | via slow or controlled release |
| IL-15 | 10 | μg/Kg- | 24 to 48 hours after induction of |
| | 10 | mg/Kg | tumor cell death to stimulate NK |
| | | | and/or proliferation or |
| | | | activation of CTL or helper cells |
| CD40L | 10 to 200 | μg/kg | 0 to 48 hours after induction of |
| | | | tumor cell death to stimulate |
| | | | maturation of DC and/or |
| | | | activation of CTL or when the |
| | | | number of DCs peaks if used as a |
| | | | tumor killing agent |
| RANKL | 10 to 200 | μg/kg | 24 to 48 hours after induction of |
| | | | tumor cell death to stimulate |
| | | | maturation of DC and/or activation |
| | | | of CTL or when the number of DCs |
| | | | peaks if used as a tumor killing agent |

Administration of a DC mobilization or maturation factor or T cell enhancing factor as a local agent in or near a tumor may allow the use of agents that are not desirable for systemic use (for example, TNF), and may be used to achieve higher concentrations of various agents at the site of the tumor than could safely be achieved using systemic administration. Similarly, agents that act as attractants for DC or CTL will also be useful for administration in or near the site of the tumor. Such local administration allows concentration of effector cells at the tumor site while minimizing systemic effects.

Various means may be used to achieve localized administration, including intratumoral injection of protein, use of gene therapy techniques to induce expression of recombinant protein in or near the tumor, and use of site-specific and/or controlled release technology. Moreover, it has been found that raw DNA, when injected into a mammal, is often taken up by cells and expressed. Accordingly, DNA encoding a desired factor may be injected into or near the site of a tumor, and, when taken up by nearby cells, will result in the localized expression of the factor encoded thereby.

One type of technology that may be useful for localized administration is that utilizing hydrogel materials to achieve sustained release of a desired factor or factors, for example, photopolymerizable hydrogels (Sawhney et al., Macromolecules 26:581; 1993). Similar hydrogels have been used to prevent postsurgical adhesion formation (Hill-West et al., Obstet. Gynecol. 83:59; 1994) and to prevent thrombosis and vessel narrowing following vascular injury (Hill-West et al., Proc. Natl. Acad. Sci. USA 91:5967; 1994). Proteins can be incorporated into such hydrogels to provide sustained, localized release of active agents (West and Hubbell, Reactive Polymers 25:139; 1995; Hill-West et al., J. Surg. Res. 58:759; 1995).

Accordingly, the various factors disclosed herein can also be incorporated into hydrogels, for application to tissues for which localized administration is desirable. For example, a hydrogel incorporating a DC attractant, DC maturational factor, or CTL enhancing factor, or a combination of various such factors, can be applied to tissue after surgical removal or reduction of the tumor. Moreover, such hydrogel-based formulations may be administered by other methods that are known in the art, for example using a catheter to apply the hydrogel at a desired location in the vascular system, or by any other means by which intratumoral administration can be accomplished. Those of ordinary skill in the art will be able to formulate an appropriate hydrogel by applying standard pharmacokinetic studies, for example as discussed by West and Hubbell, supra.

Ex Vivo Culture of DC and/or CTL

Those of skill in the art will also recognize that various ex vivo culture techniques can also be employed in the present invention. A procedure for ex vivo expansion of hematopoietic stem and progenitor cells is described in U.S. Pat. No. 5,199,942, incorporated herein by reference. U.S. Pat. No. 6,017,527 describes a method of culturing and activating DC; other suitable methods are known in the art. In one aspect of the invention, ex vivo culture and expansion comprises: (1) collecting CD34+ hematopoietic stem and progenitor cells from a patient from peripheral blood harvest or bone marrow explants; and (2) expanding such cells ex vivo. In addition to the cellular growth factors described in U.S. Pat. No. 5,199,942, other factors such as Flt3L, IL-1, IL-3, RANKL and c-kit ligand, can be used.

Stem or progenitor cells having the CD34 marker constitute only about 1% to 3% of the mononuclear cells in the bone marrow. The amount of CD34+ stem or progenitor cells in the peripheral blood is approximately 10- to 100-fold less than in bone marrow. In the instant invention, cytokines such as Flt3L, GM-CSF, CD40L and IL-15 may be used to increase or mobilize the numbers of stem cells in vivo. Such cells are then obtained and cultured using methods that are known in the art (see, for example, U.S. Pat. Nos. 5,199,942, and 6,017,527).

Isolated stem cells can be frozen in a controlled rate freezer (e.g., Cryo-Med, Mt. Clemens, Mich.), then stored in the vapor phase of liquid nitrogen using dimethylsulfoxide as a cryoprotectant; this technique will be particularly useful when the agent that induces tumor cell death is administered over time, for example, as for certain chemotherapy regimens. A variety of growth and culture media can be used for the growth and culture of dendritic cells (fresh or frozen), including serum-depleted or serum-based media. Useful growth media include RPMI, TC 199, Iscoves modified Dulbecco's medium (Iscove, et al., F. J. Exp. Med., 147:923 (1978)), DMEM, Fischer's, alpha medium, NCTC, F-10, Leibovitz's L-15, MEM and McCoy's.

The collected CD34+ cells are cultured with suitable cytokines, for example, as described herein, and in the aforementioned patents. CD34+ cells then are allowed to differentiate and commit to cells of the dendritic lineage. These cells are then further purified by flow cytometry or similar means, using markers characteristic of dendritic cells, such as CD1a, HLA DR, CD80 and/or CD86. Purified dendritic cells may pulsed with (exposed to) a desired antigen (for example, a purified antigen that is specific for the tumor at issue, a crude tumor antigen preparation or DNA or RNA encoding a tumor antigen or antigens), to allow them to take up the antigen in a manner suitable for presentation to other cells of the immune systems.

Antigens are classically processed and presented through two pathways. Peptides derived from proteins in the cytosolic compartment are presented in the context of Class I MHC molecules, whereas peptides derived from proteins that are found in the endocytic pathway are presented in the context of Class II MHC. However, those of skill in the art recognize that there are exceptions; for example, the response of CD8+ tumor specific T cells, which recognize exogenous tumor antigens expressed on MHC Class I. A review of MHC-dependent antigen processing and peptide presentation is found in Germain, R. N., Cell 76:287 (1994).

Numerous methods of pulsing dendritic cells with antigen are known; those of skill in the art regard development of suitable methods for a selected antigen as routine experimentation. In general, the antigen is added to cultured dendritic cells under conditions promoting viability of the cells, and the cells are then allowed sufficient time to take up and process the antigen, and express or present antigen peptides on the cell surface in association with either Class I or Class II MHC, a period of about 24 hours (from about 18 to about 30 hours, preferably 24 hours). Dendritic cells may also be exposed to antigen by transfecting them with DNA encoding the antigen. The DNA is expressed, and the antigen is presumably processed via the cytosolic/Class I pathway. Additionally, DC can be induced to present tumor antigen by contacting them with mRNA amplified from tumor cells, for example, as described by Boczkowski et al., Cancer Res. 60:1028, 2000.

After antigen has been processed, the DC are contacted with a DC maturation factor such as CD40L. CD40L and other DC maturation factors increase the numbers of MHC molecules (and costimulatory molecules such as CD80 and CD83) on the surface of the DC, thereby enhancing their antigen-presenting ability. Moreover, DC that have been exposed to maturation factors secrete cytokines that are indicative of activation (for example, IL-12, IL-15). CD4+cells that are presented antigen by mature, activated DC will express IL-2, IL-4, and IFN-γ, which act as growth factors for T cells. Accordingly, mature, activated DC are able to stimulate an effective, tumor-specific immune response.

Smaller antigens such as peptides do not require processing by the dendritic cell, but are bound to the appropriate MHC molecules upon exposure of the DC to the peptides. When a peptide antigen is used, it is advantageous to stimulate the maturation of the DC prior to exposure to the peptide antigen, in order to increase the numbers of available MHC molecules, and thereby enhance antigen-carrying capacity. The same DC maturation factors that are useful in stimulating the maturation of DC that have processed larger protein antigens will also be useful in augmenting the capacity of DC to present smaller peptide antigens.

The activated, antigen-carrying DC are then administered to an individual in order to stimulate an antigen-specific immune response. The DC may be administered systemically, or they may be administered locally into or near the tumor. If it is desired, additional agents such as CTL enhancing factors can be administered to the individual to further enhance the immune response. The DC can be administered prior to, concurrently with, or subsequent to, administration of additional agents. Alternatively, T cells may also be collected from the individual, and exposed to the activated, antigen-carrying dendritic cells in vitro to stimulate development of antigen-specific T cells ex vivo, which are then administered to the individual. The T cells may be administered systemically, or they may be administered locally into or near the tumor. If it is desired, T cell enhancing factors can be administered to the individual to further enhance the immune response. The T cells can be administered prior to, concurrently with, or subsequent to, administration of additional agents.

Prevention or Treatment of Disease

These results presented herein indicate that combination therapy may be of significant clinical use in the treatment of various tumors. The term treatment, as it is generally understood in the art, refers to initiation of therapy after clinical symptoms or signs of disease have been observed. However, cancer is often preceded by abnormal growth of cells that may not be strictly characterized as malignant. For example, the cells may exhibit hyperplasia, increasing in numbers but not being significantly different from normal cells of the same tissue origin. Epithelial or connective tissue cells may become metaplastic, meaning that one type of fully-differentiated cell substitutes for another. Dysplasia, in which cells lose uniformity and architectural orientation and exhibit other abnormal characteristics, frequently precedes cancer. Accordingly, the present invention will be useful in the treatment of precancerous conditions (for example, cervical intraepithelial neoplasia), the prevention or reduction of metastatic disease and prevention of relapse or reoccurence of the cancer by maximizing the potential immune response. When employed in this manner, the inventive methods described herein may be thought of as preventative measures rather than strictly defined treatment of an afflicted individual.

The relevant disclosures of all references cited herein are specifically incorporated by reference. The following examples are intended to illustrate particular embodiments, and not limit the scope, of the invention. Those of ordinary skill in the art will readily recognize that additional embodiments are encompassed by the invention.

### EXAMPLE 1

This example describes the effects of radiation therapy in combination with Flt3L and CD40L on the mean and median survival times of mice inoculated with tumor cells. Six to eight week old C57BL/6 mice were inoculated with about 1×10⁵ highly metastatic, poorly immunogenic Lewis lung carcinoma (3LL/D122) cells subcutaneously in the foot, substantially as described in Chakravarty et al. (Cancer Research 59:6028; 1999). Three weeks after inoculation, all mice had developed primary footpad tumors with pre-emergent micrometastatic foci in the lungs.

The mice were subjected to conal radiation by placing them into a lucite jig with lead body protection. A 40 MCG Philips orthovoltage unit operating at 320 kVp, 5 mA and 0.5 mm Cu filtration was used to locally irradiate the footpad are. The time at which radiation was performed was referred to as Day 0. A subset of mice were given Flt3L (10 μg per mouse intraperitoneally on each of days 1 through 12); a subset was given CD40L (10 μg per mouse intraperitoneally on each of days 8 through 12), and a subset was given Flt-3L on days 1 through 12, and CD40L on days 8 through 12 (same dosage as given previously). Results are shown in Table 2 below.

TABLE 2
Survival of Mice Treated with Combination Therapy

| | Median | Mean | | # sur- |
|---|---|---|---|---|
| Experimental | Survival | Survival | | viving/ |
| Group | (davs) | (davs) | Log Rank Test | Total |
| RT Alone | 54 | 54. | | 0/14 |
| RT + Flt3L | 151.5 | 116 | RT vs. RT + Flt3L: | 9/16 |
| | | | p = 0.00033 | |
| RT + Flt3L + | 153 | 135 | RT vs. RT + Flt3L + | 11/16 |
| CD40L | | | CD40L: p = 0.00001 | |
| RT + CD40L | 68 | 94 | RT vs. RT + CD40L: | 4/12 |
| | | | p = 0.00210 | |
| | | | RT + CD40L vs. RT + | |
| | | | Flt3L + CD40L: | |
| | | | p = 0.03 | |

These results indicate that the combination of a DC mobilization factor and a DC maturation factor augment the antitumor response of mammals subjected to radiation therapy. Accordingly, this and similar techniques will also find use ex vivo. DC may be obtained from a tumor bearing subject as described below, and exposed to tumor antigen (for example, irradiated tumor cells removed during tumor resection, or any other form of tumor antigen). The DC are exposed to a maturation agent or factor (i.e., CD40L) either before (for peptide antigens) or after processing (for large or complex antigens that require processing) to increase the numbers of MHC and costimulatory molecules and enhance antigen presentation.

The antigen-presenting DC may be administered to the tumor bearing individual, alone or in combination with T cell growth factors, resulting in enhanced ability to clear residual tumor cells (including metastases or foci of tumor burden that are not accessible to surgery or other traditional means of tumor removal). Alternatively or additionally, tumor-specific T cells can be obtained ex vivo as described below, and administered to the tumor-bearing subject, along with the DC, and/or T cell growth factors.

### EXAMPLE 2

This example describes a method for generating purified dendritic cells ex vivo. Human bone marrow is obtained, and cells having a CD34+ phenotype are isolated and cells are cultured in a suitable medium, for example, McCoy's enhanced media, that contains cytokines that promote the growth of dendritic cells (i.e., 20 ng/ml each of GM-CSF, IL-4, TNF-a, or 100 ng/ml Flt3L or c-kit ligand, or combinations thereof). The culture is continued for approximately two weeks at 37° C. in 10% CO₂ in humid air. Cells then are sorted by flow cytometry using antibodies for CD1a+, HLA-DR+ and CD86+. A combination of GM-CSF, IL-4 and TNF-α can yield a six to seven-fold increase in the number of cells obtained after two weeks of culture, of which 50-80% of cells are CD1a+ HLA-DR+ CD86+. The addition of Flt3L and/or c-kit ligand further enhances the expansion of total cells, and therefore of the dendritic cells. Phenotypic analysis of cells isolated and cultured under these conditions indicates that between 60-70% of the cells are HLA-DR+, CD86+, with 40-50% of the cells expressing CD1a in all factor combinations examined.

### EXAMPLE 3

This example describes a method for collecting and expanding dendritic cells from an individual afflicted with a tumor. Prior to cell collection, Flt3L, alone or in combination with sargramostim (GM-CSF; Leukine”, Immunex Corporation, Seattle, Wash.) is administered to an individual to mobilize or increase the numbers of circulating PBPC and PBSC. Other growth factors such as CSF-1, GM-CSF, c-kit ligand, G-CSF, EPO, IL-1, IL-2, IL-3, IL-4, IL-5, IL-6, IL-7, IL-8, IL-9, IL-10, IL-11, IL-12, IL-13, IL-14, IL-15, GM-CSF/IL-3 fusion proteins, LIF, FGF and combinations thereof, can be likewise administered separately, sequentially, or simultaneously, with Flt3L.

Mobilized PBPC and PBSC are collected using apheresis procedures known in the art. See, for example, Bishop et al., Blood, vol. 83, No. 2, pp. 610-616 (1994). Briefly, PBPC and PBSC are collected using conventional devices, for example, a Haemonetics Model V50 apheresis device (Haemonetics, Braintree, Mass.). Four-hour collections are performed typically no more than five times weekly until approximately 6.5×10⁸ mononuclear cells (MNC)/kg individual are collected.

Aliquots of collected PBPC and PBSC are assayed for granulocyte-macrophage colony-forming unit (CFU-GM) content. Briefly, MNC (approximately 300,000) are isolated, cultured at 37° C. in 5% CO₂ in fully humidified air for about two weeks in modified McCoy's 5A medium, 0.3% agar, 200 U/ml recombinant human GM-CSF, 200 u/ml recombinant human IL-3, and 200 u/ml recombinant human G-CSF. Other cytokines, including Flt3L or GM-CSF/IL-3 fusion molecules (PIXY 321), may be added to the cultures. These cultures are stained with Wright's stain, and CFU-GM colonies are scored using a dissecting microscope (Ward et al., Exp. Hematol., 16:358 (1988). Alternatively, CFU-GM colonies can be assayed using the CD34/CD33 flow cytometry method of Siena et al., Blood, Vol. 77, No. 2, pp 400409 (1991), or any other method known in the art.

CFU-GM containing cultures are frozen in a controlled rate freezer (e.g., Cryo-Med, Mt. Clemens, Mich.), then stored in the vapor phase of liquid nitrogen. Ten percent dimethylsulfoxide can be used as a cryoprotectant. After all collections from the individual have been made, CFU-GM containing cultures are thawed and pooled, then contacted with Flt3L either alone, sequentially or in concurrent combination with other cytokines listed above to drive the CFU-GM to dendritic cell lineage. The dendritic cells are cultured and analyzed for percentage of cells displaying selected markers as described above.

### EXAMPLE 4

This example illustrates the ability of dendritic cells to stimulate antigen-specific proliferation of T cells. Cells are obtained from an individual afflicted with a tumor substantially as described in Examples 2 and/or 3. Dendritic cells are isolated, and cultured for two weeks in the presence of selected cytokines. A tumor antigen preparation is made, and the dendritic cells are presented with the antigen and allowed to process it. The antigen-pulsed dendritic cells are cultured for an additional 24 hours in the presence or absence of a soluble trimeric form of CD40L (1 μg/ml) in McCoy's enhanced media containing cytokines that support the growth of dendritic cells, then pulsed with tumor antigen (Mody et al., J. Infectious Disease 178:803; 1998), at 37° C. in a 10% CO₂ atmosphere for 24 hours. Alternatively, if the tumor antigen is a small peptide that does not require processing by the dendritic cell, the dendritic cells are cultured with CD40L prior to antigen exposure. This will increase the number of HLA molecules on the dendritic cell surface, and enhance their antigen presenting capacity.

Autologous tumor-reactive T cells are derived by culturing CD34-cells from the individual in the presence of tumor antigen and low concentrations of IL-2 and/or IL-7 and/or IL-15 (2 ng/ml to 5 ng/ml) for about two weeks. The CD34-population contains a percentage of T cells (about 5%), a proportion of which are reactive against the tumor, as well as other cell types that act as antigen presenting cells. By week 2, the population of cells will comprise about 90% T cells, the majority of which will be tumor-specific, with low levels of the T cell activation markers.

Antigen specific T cell proliferation assays are conducted with the tumor-specific T cells, in RPMI with added 10% heat-inactivated fetal bovine serum (FBS), in the presence of the antigen-pulsed dendritic cells, at 37° C. in a 10% CO₂ atmosphere. Approximately 1×10⁵ T cells per well are cultured in triplicate in round-bottomed 96-well microtiter plates (Corning) for five days, in the presence of a titrated number of dendritic cells. The cells are pulsed with 1 mCi/well of tritiated thymidine (25 Ci/nmole, Amersham, Arlington Heights, Ill.) for the final four to eight hours of culture. Cells are harvested onto glass fiber discs with an automated cell harvester and incorporated cpm were measured by liquid scintillation spectrometry.

### EXAMPLE 5

This example describes the effects of antibody Ox40 m5 with or without Flt3L on the ability of mice to reject a challenge of fibrosarcoma cell in a murine model of fibrosarcoma substantially as described in Lynch et al., Eur. J. Immunol. 21:1403 (1991). Six to eight week old C57BL/10J (B10) mice were inoculated with about 1×10⁵ B10 fibrosarcoma cells subcutaneously in the foot,. Therapy with either Flt3L (10 μg per mouse intraperitoneally on each of days 10 through 29), Ox40 m5 (10 μg per mouse intraperitoneally every third day from days 10 through 27), or both, was initiated ten days after inoculation. All control mice developed tumors, as did 80% of mice given Flt3L alone, whereas 30% of mice treated with Ox40 m5 and 50% of mice treated with Ox40 m5 plus Flt3L rejected their tumors.

A similar experiment was done with another fibrosarcoma, referred to as 87, in C3H mice, utilizing two different doses of Ox40 m5 (either 100 μg per mouse or 500 μg per mouse), given on days 5, 9, 11 and 13. With the higher dose (500 μg per mouse), 40% of mice rejected the tumors, while 30% of the mice given the lower dose rejected their tumors. When Ox40 m5 was given in combination with 4-1BBm6 using substantially the same parameters, 100% of the mice given both antibodies rejected the tumor, while 60% that received 4-1BBm6 alone rejected tumor challenge.

The combination of Ox40 m5 and 4-1BBm6 was also investigated in a renal cell carcinoma model. This combination, alone or with the addition of Flt3L, did not yield significant protection from tumor challenge (only 10% of mice rejected tumor challenge), however, tumor growth was slower in animals treated with either Ox40 m5 and 4-1BBm6 or Ox40 m5, 4-1BBm6 and Flt3L. The renal carcinoma cell used are known to generate a rapidly growing tumor; accordingly, the combination of Ox40 m5 and 4-1BBm6 may prove useful even when the tumor is known to be very aggressive if given in combination with other therapy that affects the growth of the tumor.

### EXAMPLE 6

This example illustrates the ability of OX40 agonist Ox40 m5 to increase CD8 T cell activation induced by dendritic cells. A small but detectable number of naive cells from OVA-specific CD8 transgenic mice (OT.I) was transferred intravenously into naive recipients. One day after transfer, the animals were immunized subcutaneously in the hind footpads with 3×10⁵ mature dendritic cells (from Flt3L treated wild-type or MHC Class II knockout animals) pulsed with the class I OVA peptide. On the same day, the animals were also injected intraperitoneally with Ox40 m5 (100 μg) or a control monoclonal antibody. T cell expansion in the draining lymph node was monitored by FACS five days after immunization.

Co-injection of Ox40 m5 and OVA peptide-pulsed wild type dendritic cells (but not dendritic cells from class I knockout mice) strongly enhanced the CD8 T cell expansion. Lymph node cells from these immunized animals were also restimulated in vitro with the antigen. The supernatants from these cultures were assessed for IFN-γ production. Co-immunization with wild-type dendritic cells and Ox40 m5 enhanced production of IFN-γ as compared to immunization with dendritic cells alone. Lymph node cells from mice immunized with class I knockout dendritic cells produced low levels of IFN-γ upon restimulation in vitro, and the co-injection of Ox40 m5 did not enhance this production. These data indicate that OX40 agonists enhance CD8 T cell expansion and activation in vivo, and thus enhance an antigen-specific effector T cell response.

What is claimed is:

1. A method for treating a tumor-bearing subject comprising the steps of:
(a) administering a therapeutically effective amount of a dendritic cell mobilization factor to the subject; and
(b) administering a therapeutically effective amount of a tumor killing agent that stimulates maturation of dendritic cells to the subject.

2. The method of claim 1, wherein the dendritic cell mobilization factor is Flt3L, and the tumor killing agent that stimulates maturation of dendritic cells is CD40L.

3. A method for treating a tumor-bearing subject comprising the steps of:
(a) administering a therapeutically effective amount of a dendritic cell mobilization factor to the subject;
(b) administering a therapeutically effective amount of a tumor killing agent to the subject; and
(c) administering a therapeutically effective amount of a dendritic cell maturation agent to the subject.

4. The method of claim 1, wherein the dendritic cell mobilization factor is Flt3L, and the dendritic cell maturation agent is CD40L.

5. The method of any one of claims 1 through 4, wherein a T cell enhancing factor is administered in conjunction with the dendritic cell maturation agent.

6. The method of claim 5, wherein the T cell enhancing factor is Interleukin-15.

7. The method of claim 5, wherein the T cell enhancing factor is selected from the group consisting of:
(a) Ox40 agonists;
(b) 4-1BB agonists; and
(c) combinations of Ox40 agonists and 4-1BB agonists.

8. A method for treating a tumor-bearing subject comprising the steps of:
(a) administering a therapeutically effective amount of a dendritic cell mobilization factor to the subject; and
(b) treating the subject with cryotherapy.

9. The method of claim 8, wherein the dendritic cell mobilization factor is Flt3L.

10. The method of claim 8 or claim 9, wherein a dendritic cell maturation agent is administered to the tumor-bearing subject.

11. The method of claim 10, wherein a T cell enhancing factor is administered in conjunction with the dendritic cell maturation factor 5.

12. The method of claim 11, wherein the dendritic cell maturation agent is CD40L, and the T cell enhancing factor is selected from the group consisting of:
(a) Ox 40 agonists;
(b) 4-1BB agonists;
(c) combinations of Ox40 agonists and 4-1BB agonists; and
(d) Interleukin-15.

13. The method of anyone of claims 1 through 12, wherein a dendritic cell attractant is administered to attract dendritic cells to a tumor site.

14. The method of anyone of claims 1 through 12, wherein a T cell attractant is administered to attract T cells to a tumor site.

15. A method for treating a tumor-bearing subject comprising the steps of:
(a) administering a therapeutically effective amount of a dendritic cell mobilization factor to the subject;
(b) obtaining dendritic cells from the individual and culturing the dendritic cells ex vivo;
(c) administering a tumor killing agent to the individual; and
(d) administering the dendritic cells to the individual.

16. The method of claim 15, wherein the dendritic cells are contacted with a dendritic cell maturation agent ex vivo.

17. The method of claim 16 wherein the dendritic cells are contacted with an antigen prior to being contacted with the dendritic cell maturation agent.

18. The method of claim 16 wherein the dendritic cells are contacted with an antigen after being contacted with the dendritic cell maturation agent.

19. The method of any one of claims 16 through 18, wherein the dendritic cell mobilization factor is Flt3L, and the dendritic cell maturation agent is CD40L.

20. A method for treating a tumor-bearing subject comprising the steps of:
(e) administering a therapeutically effective amount of a dendritic cell mobilization factor to the subject;
(f) obtaining dendritic cells from the individual and culturing the dendritic cells ex vivo;
(g) causing the dendritic cells to become mature and active and express antigen;
(h) obtaining T cells from the individual;
(i) contacting the T cells ex vivo with the mature, active, antigen-expressing dendritic cells to obtain activated, antigen-specific T cells; and
(j) administering the activated, antigen-specific T cells to the individual.

21. The method of claim 20 wherein a T cell enhancing agent is administered to the individual before the T cells are obtained from the individual.

22. The method of claim 20 or claim 21 wherein a T cell enhancing agent is administered to the individual in conjunction with the activated, antigen-specific T cells.

23. The method of claim 22, wherein the T cell enhancing factor is selected from the group consisting of:
(a) Ox 40 agonists;
(b) 4-1BB agonists;
(e) combinations of Ox40 agonists and 4-1BB agonists; and
(f) Interleukin-15.

24. The method of anyone of claims 20 through 23, wherein a T cell attractant is administered to attract T cells to a tumor site.


SEQUENCE LISTING (6 sequences)

SEQ ID NO: 1 - protein, 235 aa, Homo sapiens
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 2 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 4 - protein, 33 aa, Artificial Sequence
  Zipper peptide
RMKQIEDKIEEILSKIYHIENEIARIKKLIGER

SEQ ID NO: 5 - protein, 281 aa, Homo sapiens [sequence omitted]

SEQ ID NO: 6 - protein, 317 aa, Homo sapiens
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID